(19) United States
(12) Patent Application Publication
(10) Pub. No.: US 2012/0265127 A1
(43) Pub. Date: Oct. 18, 2012

(54) OCCLUSION RECOGNITION IN AN ADMINISTERING APPARATUS

(76) Inventors: Thomas Buri, Burgdorf (CH); Michael Rufer, Lusslingen (CH)

(21) Appl. No.: 13/446,530

(22) Filed: Apr. 13, 2012

Related U.S. Application Data
(63) Continuation of application No. PCT/CH2010/000253, filed on Oct. 13, 2010.

(30) Foreign Application Priority Data
Oct. 16, 2009 (CH) CH 1590/09

(51) Int. Cl. A61M 5/168

(52) U.S. Cl. 604/67; 604/500

(57) ABSTRACT

A device and a method for controlling a medical administering apparatus wherein an electrical motor of the medical administering apparatus is activated during defined discharging events, including short discharging events, and the motor is controlled as per a predefined speed profile with a plurality of regions (Pᵢ), wherein a load sensor establishes load signals (Iₘₒₜ) that constitute a measure of the electrical load formed by the motor, e.g. load signals representing the motor current, wherein a monitoring arrangement compares a variable derived from the load signals with at least one predefined condition and emits an occlusion signal if the condition is satisfied, and wherein to compensate for acceleration effects, the load signals are corrected as a function of the current region by an associated correction value (I₁; I₂; I₃), thereby facilitating reliable recognition of occlusions during short discharging events.

### CROSS-REFERENCE TO RELATED APPLICATIONS

This application is a continuation of International Patent Application No. PCT/CH2010/000253 filed Oct. 13, 2010, which claims priority to Swiss Patent Application No. CH 1590/09 filed Oct. 16, 2009, the entire contents of both of which are incorporated herein by reference.

### BACKGROUND

The present invention relates to devices for injecting, delivering, administering, infusing or dispensing a substance, and to methods of making and using such devices. More particularly, it relates to a device and a method for controlling a medical administering device or apparatus which allows for an improved recognition of error states, e.g. occlusions. In some embodiments, the administering apparatus is an infusion apparatus used to administer a therapeutic substance, e.g. a fluid medicament, to a patient over a relatively long time.

In treating various diseases, it may be necessary to administer to a patient a medicament or therapeutic substance in fluid form, e.g. an insulin preparation or a blood-thinning medicament such as Heparin, over a relatively long time. Compact, portable, body-external administering apparatus or devices, which are semi-permanently or permanently carried by the patient in the vicinity of the body, are known for this purpose. For example, such an administering apparatus is disclosed in WO 2008/106806.

In such administering apparatuses, the medicament is often present in a cartridge-like reservoir, in which a piston is advanced to release or deliver the medicament from the reservoir. In some instances, the piston is advanced with the aid of an electrical drive motor, the rotational movement of which is converted into an axial advance movement of the piston via a transmission and further drive components. Typically, the administration occurs intermittently, with relatively short discharging events during which the motor is activated and hence administration occurs being separated by relatively long pauses. A complete administration cycle comprises a discharging event, which typically only takes a few seconds, and a subsequent pause, which typically lasts a few minutes.

For a reliable supply of the medicament to the patient it is essential that malfunctions or error states, which can lead to an undersupply or oversupply of the medicament, are effectively and reliably recognized and lead to the triggering of an alarm and/or the apparatus being switched off Such a malfunction or error state can result from an occlusion occurring in a liquid-carrying line, e.g. as a result of the infusion set being clogged or bent. If the drive motor is activated when an occlusion is present, this generally leads to a pressure increase in the reservoir. When the occlusion is removed, the pressure is reduced in the reservoir by the medicament flowing out of the reservoir. Thus, an occlusion can at first lead to an undersupply of the medicament, while after the removal of the occlusion, particularly one that has persisted over an extended period of time, an undesired large amount of the medicament might possibly flow out. If the medicament is, e.g., insulin, this can, in an extreme case, lead to life-threatening hypoglycaemia. It is therefore of the utmost importance that occlusions are recognized early such that the patient is alerted or warned in good time and can undertake suitable measures.

Various methods of recognizing occlusions have been proposed in the prior art, including methods based on measuring parameters of the drive motor, e.g. the electrical current flowing through the drive motor. This is based on the fact that in many types of electromotors (e.g., commercially available DC motors), the emitted torque is substantially proportional to the electrical current flowing through the motor, or at least increases continuously and monotonically with the current. If the fluid pressure increases in the reservoir, so does the force that needs to be applied for a further advance of the piston. Thus, a higher torque is required for a further advance, which in turn is expressed as an increase in the current flowing through the motor. This affords the possibility of recognizing an occlusion.

A method for recognizing occlusions based on the principle described in the previous paragraph is disclosed in US 2007/0191770 wherein the motor current is monitored and, if it exceeds a normal base-line value by a certain threshold, an occlusion alarm is triggered. However, the method presupposes that the motor has reached a state with an almost constant rotational speed (a stationary state). During the acceleration or deceleration of the motor, the inertia in the system causes an additional torque, which may falsify or falsifies the measurement. This can cause false alarms, e.g. during short discharging events in which the motor is accelerated over a significant part of the discharging event and decelerated again.

### SUMMARY

It is therefore an object of the present invention to provide a device for use in, in conjunction with and/or for controlling a medical administering apparatus, which device provides for reliable recognition of occlusions, including for short discharging events in which the motor is accelerated or decelerated over a part of the discharging event.

In one embodiment, the present invention comprises a device and a method for controlling a medical administering apparatus wherein an electrical motor of the medical administering apparatus is activated during defined discharging events and the motor is controlled as per a predefined speed profile with a plurality of regions (Pᵢ), wherein a load sensor establishes load signals (Iₘₒₜ) constituting a measure of the electrical load formed by the motor, e.g. load signals representing the motor current, wherein a monitoring arrangement compares a variable derived from the load signals with at least one predefined condition and emits a signal if the condition is met or satisfied, and wherein to compensate for acceleration effects the load signals are corrected as a function of the current region by an associated correction value (I₁; I₂; I₃).

In one embodiment, the present invention comprises a device and a method for controlling a medical administering apparatus, wherein an electrical motor of the medical administering apparatus is activated during defined discharging events, e.g. short discharging events, and the motor is controlled as per a predefined speed profile with a plurality of regions (Pᵢ), wherein a load sensor establishes load signals (Iₘₒₜ) that constitute a measure of the electrical load formed by the motor, e.g. load signals that represent the motor current, wherein a monitoring arrangement compares a variable derived from the load signals with at least one predefined condition and emits an occlusion signal if the condition is satisfied, and wherein to compensate for acceleration effects, the load signals are corrected as a function of the current region by an associated correction value (I₁; I₂; I₃) facilitating reliable recognition of occlusions even in the case of short discharging events.

In one embodiment, the present invention comprises a device for controlling a medical administering apparatus, the device comprising a motor control arrangement designed to activate an electrical motor of the medical administering apparatus during defined discharging events, the motor forming an electrical load, a load sensor for determining load signals that constitute a measure for the electrical load formed by the motor, and a monitoring arrangement designed to compare a variable derived from the load signals to at least one predefined condition and to emit an occlusion signal if the condition is satisfied, wherein the motor control arrangement is designed to control the motor with a motor speed (v) as per a predefined speed profile with a plurality of regions (P₁, P₂, P₃), and the monitoring arrangement comprises a correction module for correcting, by an associated correction value (I₁; I₂; I₃), the load signals as a function of the region (P₁; P₂; P₃) of the speed profile in which the motor control arrangement is situated.

In one embodiment, the present invention comprises an administering apparatus or device comprising a reservoir, an electrical motor for generating a drive movement, and one or more drive components coupled to the motor for transmitting the drive movement to the reservoir to eject a medicament from the reservoir, the administering apparatus or device further comprising a motor control arrangement designed to activate an electrical motor of the medical administering apparatus during defined discharging events, the motor forming an electrical load, a load sensor for determining load signals that constitute a measure for the electrical load formed by the motor, and a monitoring arrangement designed to compare a variable derived from the load signals to at least one predefined condition and to emit an occlusion signal if the condition is satisfied, wherein the motor control arrangement is designed to control the motor with a motor speed (v) as per a predefined speed profile with a plurality of regions (P₁, P₂, P₃), and the monitoring arrangement comprises a correction module for correcting, by an associated correction value (I₁; I₂; I₃), the load signals as a function of the region (P₁; P₂; P₃) of the speed profile in which the motor control arrangement is situated.

In one embodiment, the present invention comprises a method for controlling a medical administering apparatus or device comprising a reservoir for a therapeutic substance or medicament, an electrical motor forming an electrical load for generating a drive movement, and one or more drive components coupled to the motor for transmitting the drive movement to the reservoir, the method comprising the steps of activating the electrical motor during defined discharging events separated by pauses such that the drive movement generated by the motor has a motor speed substantially following a predefined speed profile with a plurality of regions (P₁, P₂, P₃), establishing load signals during the discharging events, with the load signals constituting a measure for the electrical load formed by the motor, correcting the load signals by correction values (I₁, I₂, I₃) which are dependent on the region of the speed profile in which the motor is situated, comparing a variable derived from the load signals with at least one predefined condition, and emitting an occlusion signal if the condition is satisfied or met.

Thus, according to one aspect of the present invention, a device for controlling a medical administering apparatus comprises a motor control arrangement designed to activate an electrical motor of the medical administering apparatus during defined discharging events, the electrical motor forming a (variable) electrical load for the motor control arrangement, a load sensor for determining load signals that constitute a measure for the electrical load formed by the motor, and a monitoring arrangement designed to compare a variable derived from the load signals and at least one predefined condition and to emit an occlusion signal if the condition is satisfied.

In one embodiment, the device for controlling a medical administering apparatus in accordance with the present invention is characterized by the motor control arrangement being designed to actuate the motor as per a predefined speed profile, i.e. to actuate the motor such that the motor velocity generated by the motor (more precisely: the angular velocity of the motor shaft) substantially follows the speed profile. The monitoring arrangement has a correction module for correcting, by associated region-dependent correction values, the load signals as a function of the region of the speed profile in which the motor control arrangement is actually in. This can occur before or after further processing of the load signals.

This affords the possibility of taking into account the influence of inertia on the load signals during the acceleration or deceleration of the motor. When the motor accelerates, it requires more power than if it is in a stationary state with a constant rotational speed. This leads to increased load signals. By contrast, the load signals are smaller during braking or slowing-down of the motor (i.e. during negative acceleration) than during a stationary state. The amount by which the load signals should be corrected in the case of a given acceleration or deceleration of the motor (expressed more generally, in a given region of the speed profile) can be established experimentally by measuring the load signals for the different regions of the speed profile when it is known that there is no occlusion present. These values can be established for each administering apparatus, e.g., only once during production, or when the apparatus is put into operation for the first time, or, e.g., automatically after each reservoir change, and said values can be stored as correction values in a memory of the device. The correction values can also be the same for an entire apparatus batch, be established once for the entire batch and be stored in the apparatuses of an entire batch.

In some embodiments, the load signals can constitute a measure for the electrical current flowing through the motor. Alternatively, or in addition thereto, the load signals can also represent the electrical power taken up by the motor. Other variables are also feasible as load signals, e.g. the electrical voltage across the motor, which drops at the motor at a predefined rotational speed, etc. in some embodiments, any electrical variable of the motor that allows conclusions to be drawn about the torque emitted by the motor is suitable as a load signal.

In some embodiments, the device for controlling a medical administering apparatus in accordance with the present invention can have a memory for storing the speed profile and/or regions thereof and at least one associated correction value for each region of the speed profile. The motor control arrangement is designed to read out the speed profile from the memory and actuate the motor as per the speed profile. The correction module is designed to read out the correction values from the memory and to correct the load signals accordingly.

In some embodiments, the speed profile has at least the following regions: an acceleration region in which the motor speed substantially increases, e.g. linearly with time (constant acceleration); and a deceleration region in which the motor speed substantially decreases, e.g. linearly with time (constant deceleration). In between, in some embodiments, there can be a stationary region, in which the motor speed is substantially constant; however, this stationary region can be dispensed with, e.g. in the case of very short discharging events.

In some embodiments, a first (constant) correction value for the load signals is associated with the acceleration region and a second (constant) correction value for the load signals is associated with the deceleration region. Each of these correction values is a constant, which has been established, e.g., only once already during production, when the administering apparatus is first put into operation, or after each reservoir change, and stored in the memory. By correcting the load signals of the acceleration region by a first correction value and the load signals of the deceleration region by a second correction value, it becomes possible to detect an increase in load, possibly caused by an occlusion, reliably, even if the administering events are very short such that the motor does not even reach a stationary state during the administering event.

In some embodiments, the speed profile can contain additional regions. For example, it can also be quasi-continuous, i.e. approximated by a multiplicity of successive short regions, e.g. by each of these regions being defined by a time and an associated speed value, and interpolation taking place between these speed values (sampling). It is not mandatory for the profile to contain a stationary region. Even for a general profile, the correction of the load signals allows a more reliable detection of an increased load on the motor, which could indicate an occlusion.

In some embodiments, the device for controlling a medical administering apparatus in accordance with the present invention also comprises a position sensor for establishing an actual position of the motor or a drive component connected thereto, e.g. a transmission element driven by the motor and/or a speed sensor for establishing the motor speed. In some embodiments, the sensor can be an encoder, e.g. a known optical, magnetic or other encoder, which affords the possibility of measuring both the actual position and the motor speed. Alternatively, or in addition thereto, it is also possible to use a voltage sensor as a sensor for measuring the back EMF on the motor, which allows conclusions to be drawn with respect to the motor position and/or motor speed.

This firstly affords the possibility of actively regulating the motor speed by a feedback control loop. Thus, in this case, the control arrangement has a speed controller, which receives the established motor speed as a control variable and intended speed values as per the speed profile as a reference variable and outputs a manipulated variable for the motor for regulating the motor speed as per the speed profile.

Secondly, this affords the possibility of changing the motor speed (possibly regulated by the speed controller) as a function of the motor position (rather than, e.g., as a function of time). In this case, the speed profile may be at least in part defined by virtue of the fact that intended speed values are predefined as a function of a position deviation of the actual position from an intended position. For example, the intended position can be the target position that the motor should reach at the end of a discharging event. This allows a targeted reduction in the motor speed at the end of the discharging event to avoid an overrun of the motor, after switching off the motor, without actively braking the motor (e.g. without a short-circuit brake). The speed profile can be defined by virtue of the fact that the speed profile comprises a list containing a plurality of position deviations between the actual position and an intended position, and associated intended speed values.

In some embodiments, to reduce noise and improve the reliability of occlusion recognition, a number of measures can be taken independently or cumulatively. Firstly, it is possible for a low-pass filter to be provided for the load signals to filter out fast changes in the load signals. Secondly, the monitoring arrangement may comprise an averaging module for forming averages from the load signals. The term “average” should be understood in broad terms in this case and should also comprise the (weighted or unweighted) sum of a plurality of load signals at different times. The averaging can be brought about by virtue of the fact that the load signals are registered by sampling, e.g. at predefined times or positions of the motor, e.g. after each encoder step, and the sampled load signals are summed over a certain region of samples. Up to a constant factor (the number of samples), this sum corresponds to the arithmetic mean of the samples. In the case of weighted averaging, the load signal samples are additionally multiplied by different weighting factors before the summation.

In some embodiments, the averaging module can be designed to generate the averages in a two-stage method. In a first step, a first average for successive discrete movement regions of the motor (e.g. for a predefined number of revolutions of the motor) is respectively formed in a first submodule. In a second step, second averages are formed in a second submodule by a running average from the first averages.

In some preferred embodiments, the load sensor is a current sensor for establishing load signals representing a current flowing through the motor. The current sensor can be implemented as a resistor connected in series with the motor, with an amplifier for a voltage drop across the resistor. Alternatively, or in addition thereto, the current sensor can comprise a Hall sensor for measuring a magnetic field generated by the current. Alternatively, or in addition thereto, it is also feasible e.g. to measure other electrical parameters of the motor, e.g. the voltage drop across the motor or the electrical power taken in by the motor.

In some embodiments, a device for controlling a medical administering apparatus in accordance with the present invention can additionally comprise an alarm arrangement for emitting one or more of the following signals as a function of the occlusion signals: an optical alarm signal, an acoustic alarm signal and/or a tactile alarm signal.

In some embodiments, to increase operational reliability, a device for controlling a medical administering apparatus in accordance with the present invention can comprise a redundant design, e.g. a second independent monitoring arrangement designed to compare a variable derived from the load signals with at least one predefined condition and to emit an occlusion signal if the condition is satisfied. The second monitoring arrangement may substantially have the same design as the first monitoring arrangement and may, for this purpose, have a correction module for reading out correction values from the same or from a second independent memory and for correcting, by the associated correction value, the load signals as a function of the region of the speed profile in which the motor control arrangement is situated.

In various embodiments, the monitoring arrangements, the control arrangement and the associated modules may be implemented as analogue or digital hardware or in software/firmware. For example, an analogue-digital converter (ADC) may be available for the load signals for this purpose, and the motor may be actuated in a digital fashion at the output of the motor control arrangement, e.g. via a well-known pulse-width modulator.

Some embodiments of the present invention comprise a medical administering apparatus, e.g. an infusion apparatus, for administering a fluid medicament from a reservoir, equipped with a control device of the aforementioned type. Such an administering apparatus may comprise an electrical motor interacting with the control device for generating a drive movement, and one or more drive components, coupled to the motor, for transmitting the drive movement onto the reservoir to eject or force a medicament from a reservoir. In some preferred embodiments, the motor is a DC motor.

Other embodiments of the present invention comprise a method for controlling a medical administering apparatus wherein the administering apparatus may comprise a reservoir for a fluid medicament, an electrical motor for generating a drive movement, the motor forming an electrical load, and one or more drive components, coupled to the motor, for transmitting the drive movement onto the reservoir. In one embodiment, a method in accordance with the present invention comprises the following steps:

activating the electrical motor during defined discharging events separated by pauses such that the drive movement generated by the motor has a motor speed substantially following a predefined speed profile with a plurality of regions;

establishing load signals during the discharging events, with the load signals constituting a measure for the electrical load formed by the motor;

correcting the load signals by correction values which are dependent on the region of the speed profile in which the motor is situated;

comparing a variable derived from the load signals with at least one predefined condition; and

emitting a signal if the condition is satisfied.

The same considerations in respect of the method apply analogously as in respect of the device for controlling an administering apparatus. For example, the speed profile can, as described above, have at least one acceleration region, one deceleration region and optionally one stationary region, with appropriate correction values being associated with the acceleration region and the deceleration region. However, the speed profile can also be more complex, e.g. with further regions.

As already described above, the speed profile can at least in part be prescribed by intended speed values as a function of a position deviation of the actual position from an intended position. In this instance, a method in accordance with the present invention comprises the following steps:

establishing the actual position of the motor, and

determining intended speed values as per the speed profile as a function of a position deviation of the actual position from an intended position.

In some embodiments, the motor speed can, as described above, be regulated actively with feedback. For this, the following steps are performed:

establishing the motor speed; and

regulating the motor speed as per the speed profile by a feedback control loop which receives the established motor speed as a control variable and intended speed values as per the speed profile as a reference variable and outputs a manipulated variable for the motor.

In some preferred embodiments, the load signals represent the electrical current flowing through the motor.

As described above, averages or sums over a certain number of samples can be formed from the load signals, e.g. in the aforementioned two-stage manner, with the following steps:

forming a first average over a discrete movement region of the motor in each case; and

forming second averages from the first averages by means of a running average.

In some embodiments; an occlusion signal can be emitted if at least one of the following conditions is satisfied:

a variable derived from the load signals exceeds a predefined absolute value; and

the difference between two variables derived from the load signals at different times exceeds a predefined difference value.

### BRIEF DESCRIPTION OF THE DRAWINGS

FIG. 1 is a schematic sectional illustration of an embodiment of a reusable base unit of an embodiment of a medical administering apparatus in accordance with the present invention;

FIG. 2 depicts an embodiment of a disposable cartridge that can be connected to the base unit of FIG. 1;

FIG. 3 is a schematic functional diagram of an embodiment of an administering apparatus according to the present invention;

FIG. 4 is a schematic illustration of an embodiment of a speed profile with three regions;

FIG. 5 is a diagram illustrating an embodiment of a definition of an end region of a speed profile as a function of a position deviation;

FIG. 6 is a functional diagram of an embodiment of a monitoring arrangement in accordance with the present invention; and

FIG. 7 is a diagram illustrating exemplary measured speed and current values.

### DETAILED DESCRIPTION

Any reference to “the invention” herein shall not be construed as a generalization, limitation or characterization of any subject matter disclosed and/or described herein and shall not be considered to be an element or limitation of the appended claims except if and/or where explicitly recited in a claim(s). With regard to fastening, mounting, attaching or connecting components, unless specifically described as otherwise, conventional mechanical fasteners and methods may be used. Other appropriate fastening or attachment methods include adhesives, welding and soldering, including with regard to an electrical system, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making embodiments and/or components thereof may be selected from appropriate materials such as metal, metallic alloys, ceramics, plastics, etc. Unless otherwise indicated specifically or by context, positional terms (e.g., up, down, front, rear, distal, proximal, etc.) are descriptive not limiting. Same reference numbers are used to denote same parts or components.

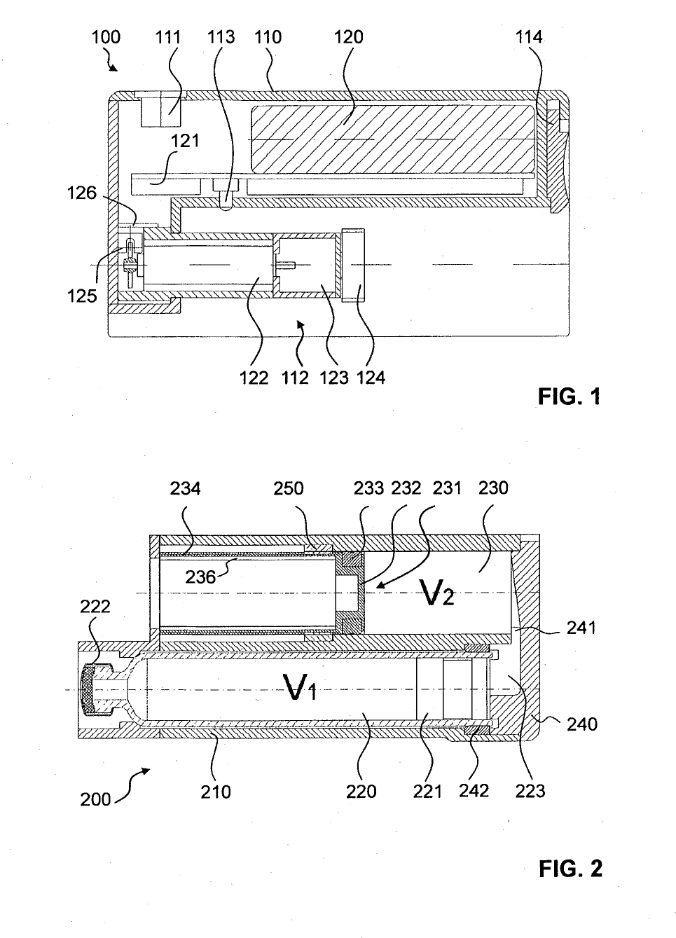

FIG. 1 illustrates in an exemplary and schematic fashion a base unit (“reusable module”) of a modular administering apparatus (“semi-disposable apparatus”) for administering a liquid medicament. A base unit having, in principle, a similar design has been described in the aforementioned international application WO 2008/106806, to which reference is made in respect of further details of the design of the base unit and the force transmission between the base unit and an associated cartridge and which is incorporated herein by reference. (WO 2008/106806 and US 2010/0152661 are owned by the owner of the present application.)

The base unit 100 has a housing 110, on or within which provision is made, inter alia, for an energy source in the form of a battery 120, an electronic control device 121 (indicated only schematically here), an electrical drive motor 122 in the form of a commercially available DC motor and further drive components including a transmission 123 and a driver 124. An encoder 125 is used to register the actual position of the drive motor in the form of the angle of rotation covered by the shaft of the drive motor and, derived from this, the motor speed. Operating elements 111 are arranged on the external side of the housing 110 and are only indicated and/or represented schematically. Such operating elements can comprise, e.g., a display as known per se, an LCD display, and one or more operating buttons. The control arrangement 121 can be operated in respect of the individual requirements of the patient by these operating elements.

The motor 122, the transmission 123 and the driver 124 are parts of a finger-like structure 112 with a substantially circular-cylindrical basic shape, with the driver being arranged in the region of the free end of this finger-like structure. The motor 122 drives the driver 124 to carry out a drive rotational movement via the transmission 123. The driver 124 comprises a wheel, on the circumferential surface of which a plurality of driving ribs are arranged extending in the axial direction. The reaction torque taken up in the process by the stator of the motor is transmitted to the housing via a suspension 126 indicated schematically in FIG. 1.

FIG. 2 illustrates a replaceable cartridge (“disposable module”), which can be connected to the base unit in FIG. 1. A suitable needle adaptor (not illustrated) may be added or operably coupled to form a complete administering device.

Directional references as indicated in the following are used to specify directions within the cartridge. The distal direction is respectively understood to be that direction in which a relevant moveable element moves during the administration of the product. In the cartridge, there is a deflection of an advance movement about 180° in the interior of the administering apparatus. The distal direction is therefore defined locally and can correspond to different absolute spatial directions in different parts of the administering apparatus. The proximal direction is in each case defined as the opposite direction to the distal direction. A lateral direction is a direction perpendicular thereto.

The cartridge 200 comprises a housing 210, which, in the region thereof illustrated at the bottom in FIG. 2, houses a product container 220 in the form of a cartridge with a cylindrical sidewall region and a product piston 221 that can be displaced therein. At its distal end, the product container 220 is closed off by an end cap 222 with a septum and thus forms a product reservoir with a volume V₁. On its proximal end, the product container is held in a sealing ring 242. A hydraulic reservoir 230 is formed antiparallel to the container 220. In the proximal direction, the hydraulic reservoir 230 is delimited by a hydraulic piston 231, which can move in the axial direction and is guided in a sealing fashion in a sidewall region of the housing. The hydraulic reservoir 230 is connected to a displacement reservoir 223 via a fluid channel 241, which is delimited by a closure element 240. Said displacement reservoir is delimited in the distal direction by the product piston 221. A suitable hydraulic fluid, for example coloured, deionised water, a suitable oil or another incompressible fluid, is filled into the hydraulic reservoir 230, into the displacement reservoir 223 and into the fluid channel 241. Overall the hydraulic fluid takes up a volume V₂.

The hydraulic piston 231 comprises a rigid support 232, on which an annular seal 233 is arranged, which seals the hydraulic piston 231 against the sidewall of the housing. The support 232 merges into a transmission sleeve 234. The transmission sleeve 234 firstly has a male thread, which engages with the female thread of a guide nut 250 arranged fixedly in the housing. Secondly, on its inner lateral face, the transmission sleeve 234 has a plurality of longitudinal grooves 236, which run parallel to the longitudinal direction of the transmission sleeve and are designed to complement the corresponding longitudinal ribs on the driver 124 of the base unit 100. While the transmission sleeve 234 is in this case designed integrally with the hydraulic piston 231, these parts can also be separate and/or rotatable with respect to one another.

To put the administering device into operational condition and subsequently administer a medicament, a needle adaptor is put on or attached to the cartridge 200, with a catheter of an infusion set connecting to the former. The infusion set ends in a cannula to puncture the skin of a patient. The needle adaptor comprises a hollow needle, which pierces the septum of the end cap 222 of the product container 220 and thus connects the interior of the product container to the catheter. Thereupon the cartridge 200 is connected to the base unit 100. In the process, the finger-like structure 112 is inserted into the interior of the transmission sleeve 234, with the longitudinal ribs on the outer side of the driver 124 engaging with the longitudinal grooves 236 in the inner lateral surface of the transmission sleeve 234. Subsequently, the cartridge 200 and the base unit 100 are locked together by a latch 114 or another suitable locking device. By a switch 113, the control arrangement determines whether the cartridge 200 is correctly connected to the base unit 100. If this is not the case, the device cannot be put into operation.

During normal operation, a certain amount of product is dispensed from the product container at predefined intervals. For this, the control device 121 actuates the motor 122, and the motor 122 sets the driver 124 into a rotational movement via the transmission 123. This rotational movement is transmitted onto the longitudinal grooves in the transmission sleeve 234 as a result of the driver 124 engaging therewith. Since the transmission sleeve 234 engages with the guide nut 250 via a threaded connection, the rotational movement at the same time brings about an advance movement of the transmission sleeve 234 (e.g. a helical movement in the distal direction) and hence an advance of the entire hydraulic piston 231 in the distal direction. This reduces the volume of the hydraulic reservoir 230, and the hydraulic fluid is pressed through the fluid channel 241 and into the displacement reservoir 223 and results in an advance of the product piston 221 in the distal direction.

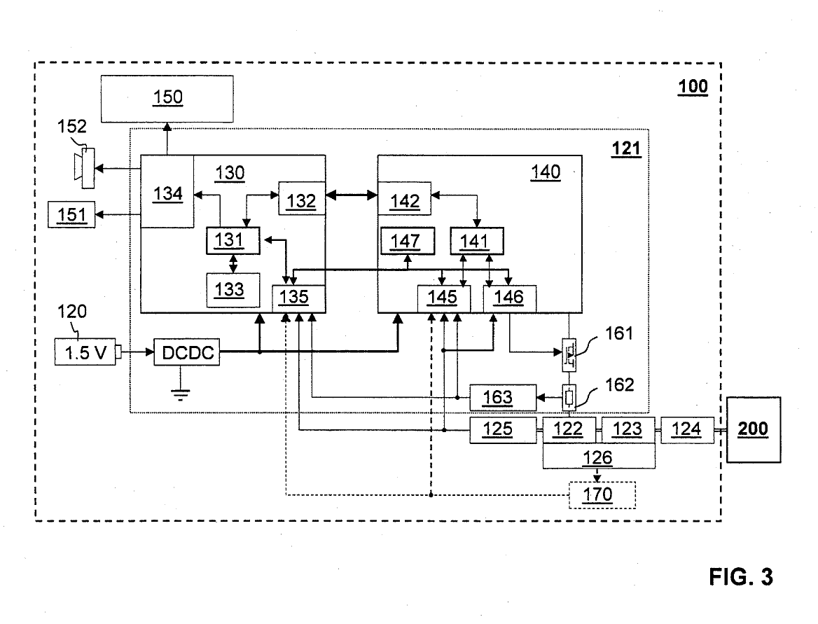

In the following, the control of such an administering apparatus shall be explained in more detail with reference to FIG. 3. Parts having the same function have been denoted with the same reference signs as in FIGS. 1 and 2. However, the type of control explained below is not limited to an administering apparatus as per FIGS. 1 and 2, but can also be used in any other administering apparatus, e.g. in infusion apparatuse or devices in which a fluid is ejected from a reservoir with the aid of an electromotor and administered to a patient.

The control device 121 comprises a digital main processor 130 and a digital supervisor processor 140, which are both supplied with voltage from the battery 120 via a DC/DC converter. The supervisor processor 140 comprising a first monitoring arrangement 145 and a motor control arrangement 146, that both interact with a first state machine 141. The state machine 141 communicates with the main processor 130 via a first interface 142. Said main processor comprises a second monitoring arrangement 135, which in turn interacts with a second state machine 131. The second state machine communicates with the first interface 142 via a second interface 132, and hence with the supervisor processor 140. Both monitoring arrangements 135, 145 communicate with a memory 147. The second state machine 131 moreover writes data to a further memory 133, which is used as an event logbook, and interacts with an alarm module 134, which can emit a tactile alarm signal via a vibrator 151, an acoustic alarm signal via a buzzer 152 and a visual (optical) alarm signal via a display 150.

The motor control arrangement 146 actuates a motor driver unit 161, which can have e.g. a MOSFET circuit, which is actuated digitally by pulse-width modulation. The motor 122 and the motor driver unit 161 are connected in series to a shunt resistor 162, across which a voltage drops, which, according to Ohm's Law, is directly proportional to the current flowing through the motor 122. This voltage is detected, amplified and low-pass filtered by a voltage detector 163 and digitized by an analogue-digital converter. The shunt resistor 162 and the voltage detector 163 form a load sensor. The monitoring arrangements 135, 145 receive digital load signals from this load sensor, which signals represent the motor current.

The motor 122 acts on the cartridge 200 via the transmission 123 and the driver 124 to eject the medicament from the product container 220 (not illustrated in FIG. 3). In the process, a reaction torque (expressed generally, a generalized reaction force), which can be registered by an optional force sensor 170, e.g. a strain gauge, acts on the suspension 126. The signals from the force sensor 170 are optionally likewise transmitted to the monitoring arrangements 135, 145, which is indicated in FIG. 3 by dashed lines.

The encoder 125 supplies position signals to the monitoring arrangements 135, 145, from which the actual position of the motor and the motor speed can be determined.

During operation, the motor control arrangement 146 intermittently activates the motor 122 at selected and/or regular intervals for a respective discharging event. There are pauses of a plurality of minutes, e.g. 5-30 minutes, between successive discharging events, while each discharging event in general takes a few seconds or a few tens of seconds. During each discharging event, a predetermined amount of the medicament is released or delivered. For this purpose, the motor is activated until it has carried out an intended number of revolutions, predetermined in advance, to correspondingly reach an end position predetermined in advance. The actual number of revolutions carried out during the discharging event is registered by the encoder 125 and compared to the intended value. Possible deviations from the intended value are compensated for in the subsequent discharging event (integral control of the released amount over successive discharging events). If the discharged amount were to drop below a minimum amount during a discharging event, the relevant discharging event is omitted, and the amount that was not discharged is likewise compensated for in the next discharging event.

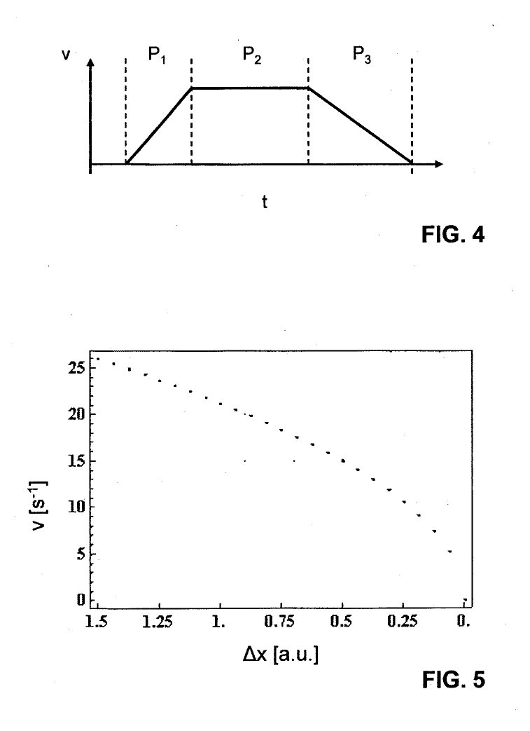

The motor is controlled in each discharging event as follows. A speed profile is stored in the memory 147. The motor control arrangement 146 reads out the speed profile from the memory 147. A simple profile of this type is illustrated schematically in FIG. 4. It comprises three phases or regions P₁, P₂, and P₃. In the region P₁, the motor speed v increases linearly with time (region of constant acceleration; start-up ramp). In the region P₂, the motor speed is constant (stationary region). Finally, in the region P₃, the motor speed reduces to zero again linearly with time (region of constant braking or slowing down, i.e. constant negative acceleration; slow-down ramp).

The motor control arrangement 146 actuates the motor as per this profile such that the motor speed substantially follows this profile. For this purpose, the motor control arrangement 146 actively regulates the motor speed by establishing the actual speed from the encoder 125 signals or a back EMF induced from the motor, and matches it to the intended speed predefined by the speed profile with the aid of a controller.

During the slow-down ramp (region P₃), the control of the motor speed is carried out dependent on position. This is illustrated in FIG. 5, which shows a typical slow-down ramp in the form of intended speed values v as a function of a deviation Ax from the desired end position. Here, the link between intended speed v and position deviation Ax is nonlinear and substantially follows a square-root function to achieve substantially constant deceleration (constant derivative of speed over time). For this purpose, a table is stored in the memory 147, which firstly contains a plurality of position deviations and secondly associated intended speed values. When the end position is approached, this table is queried by the motor control arrangement as part of the speed profile and is executed in accordance with the actual motor position. The motor speed is almost zero at the end of the discharging event. Further active braking of the motor, e.g. by short-circuiting it, can therefore be dispensed with.

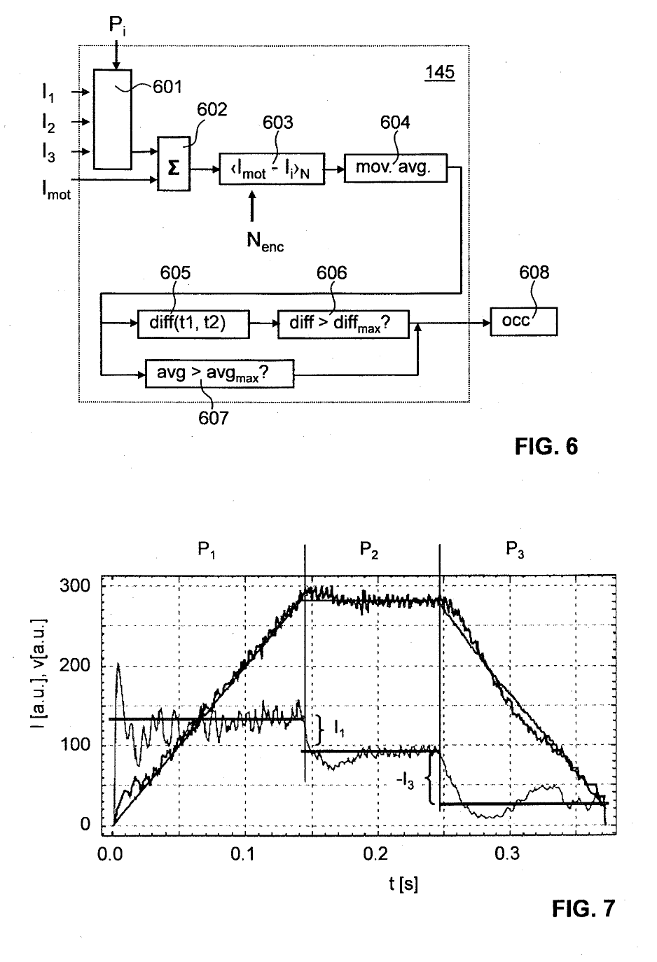

FIG. 6 illustrates the functioning of the first monitoring arrangement 145. The second monitoring arrangement may have the same or a similar design.

With the aid of signals Pᵢ, received from the motor control arrangement 146, a selection module 601 determines in which region (P₁, P₂, P₃) of the speed profile the motor 122 is situated. A current-correction value is stored in the memory 147 for each of the regions, which value was established during production before delivery of the administering apparatus and stored in the apparatus. In accordance with the region, the selection module reads out the associated current-correction value I₁, I₂=0 or I₃, and transmits the latter to a summation module 602. The latter receives the load signals emitted by the voltage detector 163, which signals represent the current I ₘₒₜ through the motor 122. In the summation module 602, the selected current-correction value Iᵢ, (i=1, 2, 3) is subtracted from these load signals in order to compensate for acceleration influences on the load signals. The difference Lₘₒₜ−Iᵢ, constitutes a good measure for the torque generated by the motor. The selection module 601 and the summation module 602 together may be thought of and/or referred to as a correction module.

The difference established by this correction module is now averaged in a two-stage method. For this purpose, a first averaging (Iₘₒₜ−Iᵢ)_{N} is carried out in a first submodule 603 over a fixed number of encoder counts N_{enc}, e.g. over a fixed (not necessarily integer) number of motor revolutions, e.g. over that number of revolutions that corresponds to a release amount of 0.1 IU. A second submodule 604 again averages these averages continuously by a moving average filter (running average). The two submodules 603, 604 together may be thought of and/or referred to as an averaging module. The moving average filter supplies an average over a number N_{enc} of encoder counts, e.g. per 0.1 IU in the present example. A predefined number of these averages is continuously stored in a results table, e.g. the last 50 averages in each case. The most recent average is firstly compared in a first comparison module 607 to a predefined maximum absolute value avgₘₐₓ for the motor current. If the average exceeds the maximum absolute value, the first comparison module 607 causes and/or emits an occlusion signal 608, since this indicates an abnormal load on the motor. Secondly, a difference between the most recent average and the oldest average in the table is formed in a differencing module 605, e.g. the increase of the corrected and averaged motor current is established over a defined or selected window. Thus, in the present example (respectively one average per 0.1 IU, 50 stored averages in the table), the increase of the motor current is established over an administered amount of 50×0.1 IU=5 IU. If this difference exceeds a certain maximum difference value diffₘₐₓ, a second comparison module 606 likewise causes and/or emits an occlusion signal 608, since this indicates a large increase in the torque generated by the motor and hence indicates a pressure increase in the container 210 or another type of blockage. In some embodiments, provision can be made for this occlusion signal to be emitted only if additionally the condition is satisfied that the most recent average exceeds a certain minimum absolute value to avoid false alarms.

Additionally, there can be further modules (not illustrated) for further monitoring functions, e.g. a measurement module for evaluating the reaction torque established by the force sensor 170. This can likewise be carried out in the manner that an occlusion signal is emitted firstly when the reaction torque exceeds a maximum absolute value and secondly when torques at different times exceed a maximum difference value.

FIG. 7 illustrates the profile of the motor speed and the motor current before being corrected and averaged, as measured for an administering apparatus of the type illustrated in FIGS. 1 and 2. As will be immediately apparent, the measured motor speed v (in arbitrary units a.u.) follows the predefined speed profile very closely. It can clearly be seen that the motor current, on average, is higher during the acceleration phase P₁ and by contrast, on average, is lower during the deceleration phase P₃ than during the stationary phase P₂. The current averages in the three phases of the speed profile are illustrated by horizontal lines in FIG. 7. The differences of the current averages in the phases P₁ and P₃ with respect to the current average in the stationary phase P₂ constitute the correction values I₁ and 1₃ used in the aforementioned method, provided the assumption is made that I₂=0. The correction values I₁, I₂ and I₃ are constant (possibly zero) if the speed changes in the associated phases are linear. This enables a correction of the dynamic error resulting from inertia.

The control of the administering apparatus as illustrated here facilitates a fast and reliable recognition of malfunctions that lead to a torque increase in the motor, e.g.

occlusions or other blockages that can lead to an interruption of the administration. Additionally, provision can optionally be made for further measures for occlusion recognition that are well-known.

A multiplicity of modifications are possible without departing from the scope of the present invention. For example, the illustrated control is not limited to the specific administering apparatus in FIGS. 1 and 2. The control need not be implemented digitally, but it can also be realized completely or in part by analogue circuits. In the case of a digital implementation, the method for controlling can be implemented as software, i.e. in a computer program product, which contains computer code carrying out the steps of the method when executed on a processor. The control of the motor speed by a speed profile with at least one acceleration region and one deceleration region can also be used independently of the aforementioned monitoring arrangement for occlusion recognition. The occlusion recognition can be used when the motor speed is controlled as per a speed profile, but not regulated actively via a feedback control loop. It is also possible to define the motor speed in the profile as a function of time instead of a position difference. A multiplicity of further modifications are possible.

Embodiments, including preferred embodiments, have been presented in this application for the purpose of illustration and description. They are not intended to be exhaustive or to limit the invention to the precise forms and steps disclosed. The embodiments were chosen and described to illustrate the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for controlling a medical administering apparatus, comprising:
a motor control arrangement to activate an electrical motor of the medical administering apparatus during defined discharging events, the motor forming an electrical load;
a load sensor for determining load signals that constitute a measure for the electrical load formed by the motor; and
a monitoring arrangement to compare a variable derived from the load signals to at least one predefined condition, and to emit an occlusion signal if the condition is satisfied; wherein
the motor control arrangement controls the motor with a motor speed as per a predefined speed profile with a plurality of regions; and
the monitoring arrangement comprises a correction module for correcting, by an associated correction value, the load signals as a function of the region of the speed profile in which the motor control arrangement is situated.

2. The device according to claim 1, wherein:
the device has a memory for storing the speed profile and in each case at least one associated correction value for each region of the speed profile,
the motor control arrangement reads the speed profile from the memory and actuates the motor as per the speed profile, and
the correction module reads the correction values from the memory and corrects the load signals accordingly.

3. The device according to claim 1, wherein the speed profile has at least an acceleration region in which the motor speed substantially increases and a deceleration region in which the motor speed substantially decreases, wherein a first correction value is associated with the acceleration region and a second correction value is associated with the deceleration region.

4. The device according to claim 1, further comprising a position sensor for establishing an actual position of the motor or of a drive component connected thereto.

5. The device according to claim 4, wherein the speed profile is at least in part defined by intended speed values predefined as a function of a position deviation of the actual position from an intended position.

6. The device according to claim 1, further comprising an arrangement for determining the motor speed, the control arrangement comprising a speed controller which receives a determined motor speed as a control variable and intended speed values as per the speed profile as a reference variable and outputs a manipulated variable for the motor for regulating the motor speed as per the speed profile.

7. The device according to claim 1, wherein the monitoring arrangement further comprises an averaging module for forming averages or sums from the load signals.

8. The device according to claim 7, wherein the averaging module comprises a first submodule for respectively forming a first average for successive discrete movement regions of the motor and a second submodule for forming second averages from the first averages by a running average.

9. The device according to claim 1, wherein the load sensor further comprises a current sensor for establishing load signals representing a current flowing through the motor.

10. The device according to claim 9, wherein the current sensor comprises a resistor connected in series with the motor and an amplifier for a voltage drop across the resistor or a Hall sensor for measuring a magnetic field generated by the current.

11. The device according to claim 1, wherein
the monitoring arrangement further comprises a first comparison module for emitting an occlusion signal when a variable derived from the load signals exceeds a predefined absolute value, a differencing module for forming differences between respectively two variables derived from the load signals at different times, and a second comparison module for emitting an occlusion signal when the difference exceeds a predefined difference value.

12. The device according to claim 11, further comprising an alarm arrangement for emitting one or more of an optical alarm signal, an acoustic alarm signal and/or a tactile alarm signal as a function of the occlusion signals.

13. The device according to claim 1, further comprising a second independent monitoring arrangement for comparing a variable derived from the load signals with at least one predefined condition and for causing an occlusion signal if the condition is satisfied.

14. A medical administering apparatus for administering a fluid medicament, the medical administering apparatus comprising:
a reservoir;
a device for controlling the medical administering apparatus, comprising a motor control arrangement to activate an electrical motor of the apparatus during defined discharging events, the motor forming an electrical load, a load sensor for determining load signals that constitute a measure for the electrical load formed by the motor, and a monitoring arrangement to compare a variable derived from the load signals to at least one predefined condition and to cause an occlusion signal if the condition is satisfied, wherein the motor control arrangement controls the motor with a motor speed as per a predefined speed profile with a plurality of regions and the monitoring arrangement comprises a correction module for correcting, by an associated correction value, the load signals as a function of the region of the speed profile in which the motor control arrangement is situated;
an electrical motor for generating a drive movement, said motor interacting with said device for controlling; and
one or more drive components coupled to the motor for transmitting the drive movement to the reservoir to eject the medicament from the reservoir.

15. A method for controlling a medical administering apparatus comprising a reservoir for a fluid medicament, an electrical motor forming an electrical load for generating a drive movement, and one or more drive components coupled to the motor for transmitting the drive movement to the reservoir, the method comprising the following steps:
activating the electrical motor during defined discharging events separated by pauses such that the drive movement generated by the motor has a motor speed substantially following a predefined speed profile with a plurality of regions;
establishing load signals during the discharging events, the load signals constituting a measure for the electrical load formed by the motor;
correcting the load signals by correction values which are dependent on the region of the speed profile in which the motor is situated;
comparing a variable derived from the load signals with at least one predefined condition; and
emitting an occlusion signal if the condition is satisfied.

16. The method according to claim 15, wherein the speed profile has at least an acceleration region in which the motor speed substantially increases and a deceleration region in which the motor speed substantially decreases, wherein a first correction value is associated with the acceleration region and a second correction value is associated with the deceleration region.

17. The method according to claim 15, wherein the correction values are established automatically when the administering apparatus is operated for the first time and/or after the reservoir has been replaced and are stored in a memory of the administering apparatus.

18. The method according to claim 15, further comprising establishing an actual position of the motor and determining intended speed values as per the speed profile as a function of a position deviation of the actual position from an intended position.

19. The method according to claim 18, further comprising establishing the motor speed and regulating the motor speed by a feedback control loop which receives the established motor speed as a control variable and intended speed values as per the speed profile as a reference variable and outputs a manipulated variable for the motor.

20. The method according to claim 15, further comprising forming averages or sums from the load signals.

21. The method according to claim 20, wherein the forming of averages comprises respectively forming a first average over a discrete movement region of the motor in each case and forming second averages from the first averages by a running average.

22. The method according to claim 15, wherein an occlusion signal is emitted if at least one of the following conditions is satisfied:
a variable derived from the load signals exceeds a predefined absolute value; and
the difference between two variables derived from the load signals at different times exceeds a predefined difference value.
